(19) United States
(12) Patent Application Publication
(10) Pub. No.: US 2005/0070509 A1
(43) Pub. Date: Mar. 31, 2005

(54) Antimycotic agent

(76) Inventors: Dieter Zeller, Speyer (DE); Jurgen Tropsch, Romerberg (DE); John-Bryan Speakman, Bobenheim (DE); Peter Neumann, Mannheim (DE); Hans-Peter Heidenreich, Sinzheim (DE)

Correspondence Address: Keil & Weinkauf, 1101 Connecticut Avenue NW, Washington, DC, 20036, US

(21) Appl. No.: 10/479,624

(22) PCT Filed: Jun. 4, 2002
(86) PCT No.: PCT/EP02/06128

(30) Foreign Application Priority Data
Jun. 5, 2001 (DE) 10127244.8

(51) Int. Cl. A61K 31/655; C07F 1/08

(52) U.S. Cl. 514/149; 534/569

(57) ABSTRACT

The present invention relates to a metal salt for use in pharmacy, to pharmaceutical compositions comprising it, and to the use for preparing a pharmaceutical composition for treating diseases associated with mycobionts.

The present invention relates to a metal salt for use in pharmacy, to pharmaceutical compositions comprising it, and to the use for preparing a pharmaceutical composition for treating diseases associated with mycobionts.

In particular, the invention relates to the use of a bis(N-organyldiazeniumdioxy) metal salt as composition for the treatment of humans and animals infected with harmful microorganisms, in particular mycobionts.

It is known that bis(N-organyldiazeniumdioxy) salts are fungicidally active. For example, DT-A 1 817 571 describes a mixture of alkali metal hydroxide and a heavy-metal salt of N-nitroso-N-cyclohexylhydroxylamine which is employed as fungicide in timber preservatives. Furthermore, DE 24 10 603 discloses a timber-protection fungicide which comprises heavy-metal salt derivatives of N-nitroso-N-cyclohexylhydroxylamine.

Although a large number of antimycotics, for example miconazole and clotrimazole (Canesten®, Bayer), are known, there is a constant demand for novel antimycotic active ingredients which have novel or broader spectra of action or which are active with respect to fungi which have developed resistances to known antimycotics.

It is an object of the present invention to provide a novel pharmaceutically active, in particular antimycotically active, compound for use as pharmaceutical which overcomes the disadvantages of the conventional compositions.

We have found that this object is achieved by a variety of bis(N-organyldiazeniumdioxy) metal salts which have potent antimycotic activity against organisms capable of causing mycoses in humans or animals.

The present invention therefore relates to a metal salt of the formula 1: 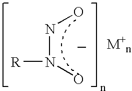
in which
R is C₁-C₆-alkyl, C₃-C₈-cycloalkyl or aryl,
M⁺ is a cation equivalent, and
n is an integer from 1 to 3,
for use as pharmaceutical.

For the purposes of the present invention, the term ‘alkyl’ encompasses straight-chain or branched alkyl groups. These are preferably straight-chain or branched C₁-C₄-alkyl groups. Examples of alkyl groups are, in particular, methyl, ethyl, propyl, isopropyl, n-butyl, 2-butyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl and 1-ethyl-2-methylpropyl.

The cycloalkyl group is preferably a C₅-C₇-cycloalkyl group such as cyclopentyl, cyclohexyl or cycloheptyl.

The aryl group is preferably phenyl or tolyl.

M⁺ is a cation equivalent, i.e. a monovalent cation, or that portion of a polyvalent cation or a positively charged metal-atom-containing group which corresponds to a single positive charge. For example, M⁺ is an alkali metal cation such as Li⁺, Na⁺ or K⁺. Suitable bivalent cations are, for example, Cu²⁺, Zn²⁺, Ni²⁺ and Co²⁺. Suitable trivalent cations are, for example, Fe³⁺ and Al³⁺. Suitable monovalent metal-atom-containing groups are, for example, tin-containing groups of the formula R^{a}R^{b}R^{c}Sn⁺ in which R^{a}, R^{b} and R^{c} independently of one another are C₁₋₆-alkyl radicals. R¹, R² and R³ are preferably butyl, i.e. the metal-atom-containing group is (C₄H₉)₃Sn⁺.

Preferred cations are K⁺, Cu²⁺ and Al³⁺. Especially preferred as metal M is copper.

Preferred radicals R are C₅- and C₆-alkyl or C₅- and C₆-cycloalkyl groups, in particular cyclohexyl.

Preferred metal salts are N-cyclohexyldiazeniumdioxypotassium and tris(N-cyclohexyldiazeniumdioxy)aluminum.

An especially preferred embodiment relates to the use according to the invention of bis(N-cyclohexyldiazeniumdioxy)copper of the formula 2:

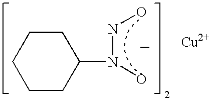

The invention furthermore relates to a pharmaceutical composition comprising at least one compound of the formula 1 as defined above and at least one or more pharmaceutically acceptable carrier(s) and/or additive(s).

The invention also relates to methods for the treatment of diseases associated with mycobionts, in which an antimycotically active amount of a compound of the formula 1 according to the invention is administered to a person or to an animal requiring such a treatment.

Mycobionts, also termed fungi or Mycota, are eucaryotic organisms which grow under aerobic conditions and obtain the energy required by oxidizing organic substances. Some representatives, for example yeasts, are facultatively viable under anaerobic conditions, obtaining their energy by fermentation processes. The representatives of the mycobionts include, for example, yeasts or budding fungi, molds, dimorphic fungi and dermatophytes.

Mycoses are diseases caused by fungi; they may occur locally or generally. They occur, inter alia, when the immune system is compromised, for example during therapies involving antibiotics or cytostatics, during the administration of steroids or hormones, following irradiation, during parenteral feeding, or during malignant diseases, endocrinopathies or immunodeficiences. In the case of systemic mycoses, specific organs are infected preferentially. Dermatomycoses, for example, are diseases where specific fungal species, in particular dermatophytes and yeasts, infect the skin and/or its cutaneous appendages. The fungi penetrate the skin, hair, hair folicles and finger- or toenails and cause symptoms such as vesiculation, exfoliation, skin fissures, and erosion, in most cases in conjunction with pruritus and/or allergic eczema. While dermatomycoses affect almost exclusively the skin, hair and nails, mycoses caused by yeasts may also spread to mucus membranes and internal organs.

Surprisingly, it has now been found that the metal salts of the formula 1, in particular the copper salts thereof, have potent antimycotic activity. An example of an especially preferred compound which can be used in accordance with the invention is bis(N-cyclohexyldiazeniumdioxy)copper. The spectrum of action of the compounds which can be used in accordance with the invention extends to yeasts, dermatophytes, molds, Pityrosporum ovale and biphasic fungi. The compounds according to the invention can therefore be employed successfully as active substance for the treatment of a large number of local and systemic mycoses in humans and animals. The active ingredients according to the invention are particularly effective in the treatment of dermatomycoses caused by Trichophyton rubrum, Trichophyton mentagrophytes and other Trichophyton species, Microsporum canis, Epidermophyton floccosum and Scopulariopsis brevicaulis, and candidoses caused by Candidai tropicalis, Candida albicans, Candida glabrata, Candida parapsilosis and further Candida species.

Diseases in which the compounds according to the invention can be employed are, for example, disorders of the immune system, HIV infections, AIDS, skin diseases, diseases of the airways and the pharynx, systemic infections, local infections, for example of the skin, the hair or the nails, infections of the mucus membranes, otitis, pharyngitis, pneumonia, pyelonephritis, cystitis, endocarditis, bronchitis and arthritis.

The present invention also includes pharmaceutical preparations comprising one or more active ingredients according to the invention and one or more nontoxic inert pharmaceutically acceptable carrier materials and, if appropriate, one or more nontoxic inert pharmaceutically acceptable adjuvants and one or more nontoxic inert pharmaceutically acceptable additives.

Pharmaceutically acceptable materials are the substances which, as is known, can be used in the pharmaceutical sector, the food technology sector and related fields, in particular the substances listed in specialist pharmacopeias and whose properties are no obstacle to physiological applications.

The bis(N-organyldiazeniumdioxy) metal salts which are employed in accordance with the invention as antimycotic active ingredients are prepared by customary methods known to the skilled worker.

For example, the active ingredients which can be used in accordance with the invention can be formulated as tablets, coated tablets, capsules, pills, granules, suppositories, solutions, suspensions and emulsions, pastes, ointments, gels, creams, lotions, powder or sprays.

Suitable carrier materials for tablets, coated tablets, capsules, pills and granules are customary fillers and extenders such as starches, lactose, sucrose, glucose, mannitol and silicic acid.

Suitable carrier materials for suppositories, solutions, suspensions, emulsions, pastes, ointments, gels, creams and lotions are selected from water, hydrophilic components and hydrophobic components, and mixtures of these. Suitable hydrophilic components in excipients are, for example, mono-, di- or polyhydric alcohols having preferably 1 to 8 carbon atoms, such as ethanol, n-propanol, isopropanol, propylene glycol, glycerol, sorbitol and the like. Suitable hydrophobic components in excipients are, for example, oily or fatty components such as solid and liquid paraffins; Vaseline; natural fats and oils such as castor oil, soya oil, corn oil, cottonseed oil, peanut oil, olive oil, sunflower oil, sesameseed oil, avocado oil, cocoa butter, almond oil, peach kernel oil, codliver oil, lard, spermaceti, spermaceti oil, sperm oil, wheatgerm oil, macadamia nut oil, oil of evening primrose, jojoba oil; fatty alcohols such as lauryl alcohol, myristyl alcohol, cetyl alcohol, stearyl alcohol, oleyl alcohol; fatty acids such as myristic acid, stearic acid, palmitic acid, oleic acid, linoleic acid, linolenic acid; waxes such as beeswax, carnauba wax, candelilla wax, spermaceti and mixtures of these.

Suitable carrier materials for powders or sprays are, for example, lactose, talc, silicic acid, aluminum hydroxide, calcium silicate and polyamide powder, or mixtures of these. Sprays may additionally comprise the customary propellants, for example, fluorochlorohydrocarbons.

The preparations according to the invention may furthermore comprise one or more nontoxic, inert pharmaceutically acceptable adjuvants. These can take the form of solid, semisolid or liquid materials which act as vehicles, carriers or constituents for the active ingredient. Examples of suitable adjuvants are lubricants, wetting agents, emulsifiers, suspending agents, preservatives, adsorbents, antioxidants, antiinflammatories, binders, chelating agents, emulsion stabilizers, humectants, film formers, gellants, odor-masking agents, resins, hydrocolloids, solvents, solubilizers, solution retardants, neutralizing agents, penetrants, pigments, quaternary ammonium compounds, resorbents, superfatting agents, excipients for ointments, creams or oils, silicone derivatives, stabilizers, sterilants, propellants, desiccants, opacifying agents, thickeners, waxes, plasticizers, white oils and other diluents, fillers and formulation auxiliaries of any type. If desired, the adjuvants and/or further additives such as odor- and/or flavor-improving additions or colorants are admixed in a manner with which the skilled worker is familiar.

The tablets, coated tablets, capsules, pills or granules can be provided with the customary coatings and coats which, if desired, comprise opacifying agents. They may also be present in microencapsulated form or be composed in such a way that they release the active ingredient(s) only, or preferentially, in a particular part of the intestinal tract, if appropriate in a sustained manner. Embedding materials which can be used in this context are, for example, polymeric substances and waxes.

For parenteral administration, solutions or emulsions which can be used in accordance with the invention may be present in sterile and blood-isotonic form.

The pharmaceutical preparations according to the invention can be formulated in a variety of dose units. The dose units may correspond, for example, to a unit dose, a fraction of a unit dose or a multiple thereof. Examples of dose units are one, two, three or four unit doses or half, a third or a quarter of a unit dose. A unit dose preferably comprises an amount of active ingredient which corresponds to a daily dose or to half, a third or a quarter thereof.

The therapeutically active compounds in the above-mentioned pharmaceutical preparations should be present in a concentration of from approximately 0.0001 to 99.5% by weight, preferably from 0.001 to 95% by weight, specifically from 0.01 to 50% by weight, based on the total mixture. Advantageously, a therapeutic activity in a variety of mycoses is evidenced even at very low active ingredient concentrations such as 0.00015% by weight. In addition to the active ingredients according to the invention, the preparations may also comprise further pharmaceutical active ingredients.

The pharmaceutical preparations according to the invention are prepared in the customary manner by method known to the skilled worker.

The present invention also includes a method of treating diseases associated with mycobionts. In this context, an antimycotically active amount of the active ingredient according to the invention is administered to a person or an animal reuqiring such a treatment. The active ingredient or the pharmaceutical preparation can be administered locally, orally, parenterally, intraperitoneally and/or rectally, preferably orally or locally.

In the case of systemic administration, it has been generally proved advantageous to administer the active ingredient(s) according to the invention in a total amount of from approximately 0.3 to 80 mg/kg body weight, preferably 3 to 15 mg/kg body weight, per 24 hours. The amount of active ingredient may be administered at once or split into several single doses. In some cases, however, it may be necessary to deviate from the abovementioned proposed dosages, viz. as a function of the body weight, the nature and severity of the disease, or the type of preparation or of pharmaceutical form. Thus, it may suffice in some cases to employ a smaller amount of active ingredient, while in other cases the abovementioned amount of active ingredient may be exceeded. The amount which is most suitable in each case can be determined readily by the skilled worker.

The invention also relates to a composition in the form of a commercial pack with at least one composition based on a metal salt as defined above, together with instructions for therapeutical use.

The invention also relates to the use of a metal salt of the formula 1 where R is C₁-C₆-alkyl, C₃-C₈-cycloalkyl or aryl, M⁺ is a cation equivalent and n is an integer from 1 to 3, for the preparation of a pharmaceutical composition for treating diseases associated with mycobionts.

If the active ingredient according to the invention is used as feed additive, it may be administered in the customary manner together with the feed or the feed product or the drinking water.

The activity of the active ingredient according to the invention as antimycotic was studied in an in-vitro agar incorporation test. To this end, various samples of mycobionts were grown in a nutrient medium of Sabouraud agar and supplemented with various amounts of active ingredient. The active ingredient concentration in the medium was in the range from 1 to 100 ppm. Incubation times were between 1 and 21 days. The experiments with yeasts of the Candida type revealed effective growth inhibition at an active ingredient concentration of 50 ppm and an incubation time of from 2 to 5 days. Experiments with dermatophyte cultures revealed effective growth inhibition at an active ingredient concentration in the range from 15 to 40 ppm after an incubation time of 1 to 3 weeks.

### EXAMPLES

### Example 1

Use of bis(N-cyclohexyldiazeniumdioxy)copper for inhibiting the growth of various Candida species.

The activity of the compound according to the invention as agent for inhibiting the growth of various types of yeasts was studied in an agar incorporation test. The nutrient medium used was Sabouraud agar. Several samples of each of the yeast types below, which were isolated directly from diseased patients, were tested:
a) Candida tropicalis
b) Candida albicans
c) Candida glabrata
d) Candida parapsilosis.

The inoculum suspensions had a density of 10⁷ colony-forming units per ml. The cultures were supplemented with bis(N-cyclohexyldiazeniumdioxy)copper in such an amount that an end concentration of 25 or 50 ppm was obtained. The cultures were subsequently incubated at 30° C., and the growth of the isolates was assessed after an incubation period of 2 to 5 days.

At an active ingredient concentration of 25 ppm, all of the isolates tested were still growing after an incubation period of 2 and 5 days. At an active ingredient concentration of 50 ppm, however, no growth took place.

### Example 2

Use of bis(N-cyclohexyldiazeniumdioxy)copper for inhibiting the growth of various species of dermatophytes.

The activity of the compound according to the invention for inhibiting the growth of the dermatophyte cultures below was studied in an agar incorporation test as described in Example 1. The active ingredient concentration in the samples amounted to 2.5, 5, 10, 15, 20, 25, 30, 35, 40, 45 and 50 ppm. The isolates were studied after an incubation period of 7, 14 and 21 days, and their growth was determined. The active ingredient concentrations at which effective growth inhibition was observed are shown in the table which follows.

| | Effective active ingredient | | |
|---|---|---|---|
| Dermatophyte | concentration [ppm] | | |
| Incubation period | 7 days | 14 days | 21 days |
| Trichlorophyton | 15 | 15 | 15 |
| rubrum | | | |
| Trichophyton | 30 | 30 | 30 |
| mentagrophytes | | | |
| Microsporum canis | ≦10 | 15 | 15 |
| Epidermophyton | 15 | 20 | 20 |
| floccosum | | | |
| Scopulariopsis | 25 | 35 | 40 |
| brevicaulis | | | |

The most effective inhibition of dermatophyte growth was observed after an incubation period of 21 days at a concentration of from 15 to 40 ppm.

1. A metal salt of the formula 1 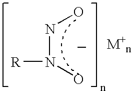
in which
r is c₁-C₆-alkyl, c₃-C₈-cycloalkyl or aryl,
M⁺ is a cation equivalent, and
n is an integer from 1 to 3,
for use as pharmaceutical.

2. A metal salt as claimed in claim 1 in which M is a bivalent metal cation selected from the group consisting of copper, zinc, nickel and cobalt.

3. A metal salt as claimed in claim 2, in which the metal cation is the copper cation.

4. A metal salt as claimed in claim 1, which is bis(N-cyclohexyldiazeniumdioxy)copper.

5. A metal salt as claimed in claim 1 for treating diseases associated with mycobionts.

6. A pharmaceutical composition comprising at least one compound of the formula 1 as defined in claim 1 and at least one or more pharmaceutically acceptable carrier(s) and/or additive(s).

7. A composition in the form of a commercial pack with at least one composition based on a metal salt as defined in claim 1, together with instructions for therapeutic use.

8. A method of treating diseases associated with mycobionts, in which an antimycotically active amount of a compound of the formula 1 as defined in claim 1 is administered to a person or to an animal requiring such a treatment.

9. The metal salt of the formula 1 in which R is C₁-C₆-alkyl, C₃-C₈-cycloalkyl or aryl, M⁺ is a cation equivalent and n is an integer from 1 to 3 for the preparation of a pharmaceutical composition for treating diseases associated with mycobionts.
